(19) United States
(12) Patent Application Publication
(10) Pub. No.: US 2026/0137823 A1
(43) Pub. Date: May 21, 2026

(54) CABINET FOR CLEANING MEDICAL DEVICES

(71) Applicant: CareFusion 303, Inc., San Diego, CA (US)

(72) Inventor: Vinay JANI, San Diego, CA (US)

(21) Appl. No.: 18/955,035

(22) Filed: Nov. 21, 2024

(51) Int. Cl. A61L 2/18; A61L 2/10; A61L 2/24

(52) U.S. Cl. CPC A61L 2/18; A61L 2/10; A61L 2/24; A61L 2103/15; A61L 2202/122

(57) ABSTRACT

The present disclosure regards a cabinet for cleaning medical devices. The cabinet includes a housing, a mechanical retainer, a robotic cleaning head, and a processor. The housing includes an enclosure for receiving one or more medical devices and a door configured to secure the enclosure. The mechanical retainer is positioned on a surface of the enclosure and configured to immovably couple the medical device to the surface while the medical device is received within the enclosure. The robotic cleaning head is movably coupled to another surface of the enclosure and configured to move therein and apply a cleaning agent to the medical device. The processor is configured to cause the cleaning head to move within the enclosure and apply the cleaning agent to clean the one or more medical devices after determining that the one or more medical devices are received within the enclosure and the door is securing it.

### TECHNICAL FIELD

The present disclosure relates, generally, to medical devices (e.g., infusion pumps) and, more specifically, to cabinets for the automated cleaning thereof.

### BACKGROUND

In healthcare, maintaining clean medical equipment is crucial for preventing infections, ensuring patient safety, and achieving accurate diagnostics. While regular cleaning is important for all non-disposable medical equipment, it is particularly crucial for infusion pumps and other devices used in infusion therapy as they are frequently exposed to fluids and contaminants.

Implementing a consistent cleaning protocol poses significant challenges. The cleaning itself is time-consuming, and the effort required to ensure that devices are cleaned both regularly and thoroughly requires additional time and resources. This is exacerbated in large healthcare facilities, such as hospitals, where hundreds or even thousands of devices require regular cleaning.

### SUMMARY

The subject technology seeks to address these issues with a cabinet for automating the cleaning of medical devices and other medical equipment. The cabinet is configured to apply a cleaning agent (e.g., a light-based agent, heightened temperatures, a chemical agent) to medical devices while the devices are stored therein. Additionally, the cabinet can be configured to perform various “smart” functions designed to improve cleaning and simplify clinician workflow.

Exemplary implementations include the following:
A Cabinet for Cleaning Medical Devices. The cabinet includes a housing, a mechanical retainer, a robotic cleaning head, and a processor. The housing includes an enclosure sized to receive one or more medical devices. The housing also includes a door configured to secure the enclosure. The mechanical retainer is positioned on a first surface of the enclosure and configured to retain the one or more medical devices while they are received within the enclosure. The robotic cleaning head is movably coupled to a second surface of the enclosure and configured to move within the enclosure and apply a cleaning agent to the one or more medical devices while they are received within the enclosure. The processor is configured to determine that the one or more medical devices are received within the enclosure, determine that the door is in a closed position to secure the enclosure, and cause the robotic cleaning head to move within the enclosure to and apply the cleaning agent to clean the one or more medical devices after determining that the one or more medical devices are received within the enclosure and the door is in the closed position.
A Computer-Implemented Method. The method is for automating cleaning of medical devices. It includes determining, by a processor of a cabinet, that one or more medical devices are received within an enclosure sized to receive the one or more medical devices. In addition to the processor, the cabinet includes a housing with the enclosure, as well as a door configured to secure the enclosure. The cabinet also includes a mechanical retainer positioned on a first surface of the enclosure and configured to immovably couple the one or more medical devices to the first surface of the enclosure while the one or more medical devices are received within the enclosure. Additionally, the cabinet includes a robotic cleaning head movably coupled to a second surface of the enclosure and configured to move within the enclosure and apply a cleaning agent to the one or more medical devices while the one or more medical devices are received within the enclosure. The method also includes determining, by the processor, that the door is in a closed position to secure the enclosure. Additionally the method includes causing, by the processor, the robotic cleaning head to move within the enclosure and apply the cleaning agent to clean the one or more medical devices after determining that the one or more medical devices are received within the enclosure and the door is in the closed position.
A Non-Transitory, Computer-Readable Storage Medium. The non-transitory, computer-readable storage medium includes instructions that, when executed by a processor of a cabinet for cleaning medical devices, cause the cabinet to determine that one or more medical devices are received within an enclosure sized to receive the one or more medical devices. In addition to the processor, the cabinet also includes a housing, a mechanical retainer, and the aforenoted cleaning head. The housing includes the enclosure and a door configured to secure the enclosure. The mechanical retainer is positioned on a first surface of the enclosure and configured to immovably couple the one or more medical devices to the first surface of the enclosure while the one or more medical devices are received within the enclosure. The robotic cleaning head is movably coupled to a second surface of the enclosure and configured to move within the enclosure and apply a cleaning agent to the one or more medical devices while the one or more medical devices are received within the enclosure. The instructions further cause the cabinet to determine that the door is in a closed position to secure the enclosure. Additionally, the instructions cause the cabinet to cause the robotic cleaning head to move within the enclosure and apply the cleaning agent to clean the one or more medical devices after determining that the one or more medical devices are received within the enclosure and the door is in the closed position.

Based on the Detailed Description below, other configurations of the subject technology will be apparent to those skilled in the art. The Detailed Description describes various configurations of the subject technology, particularly with respect to illustrations thereof. Notwithstanding, the subject technology is capable of other and different configurations, and its several details are capable of modification in various other respects - all without departing from the scope of the subject technology. The Drawings and Detailed Description are therefore presented as illustrative in nature and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIG. 1 illustrates an example cabinet for cleaning medical devices stored therein, according to various aspects of the subject technology.

FIG. 2 illustrates an example process for cleaning medical devices, according to various aspects of the subject technology.

FIG. 3 illustrates an example electronic system for cleaning medical devices, according to various aspects of the subject technology.

### DETAILED DESCRIPTION

Reference will now be made to implementations, examples of which are illustrated in the accompanying drawings. In the following description, specific details are set forth in order to provide an understanding of the various described implementations. However, it will be apparent to one of ordinary skill in the art that the various described implementations may be practiced without one or more of these details. In other instances, well-known methods, procedures, components, circuits, and networks have not been described in detail so as not to unnecessarily obscure aspects of the implementations.

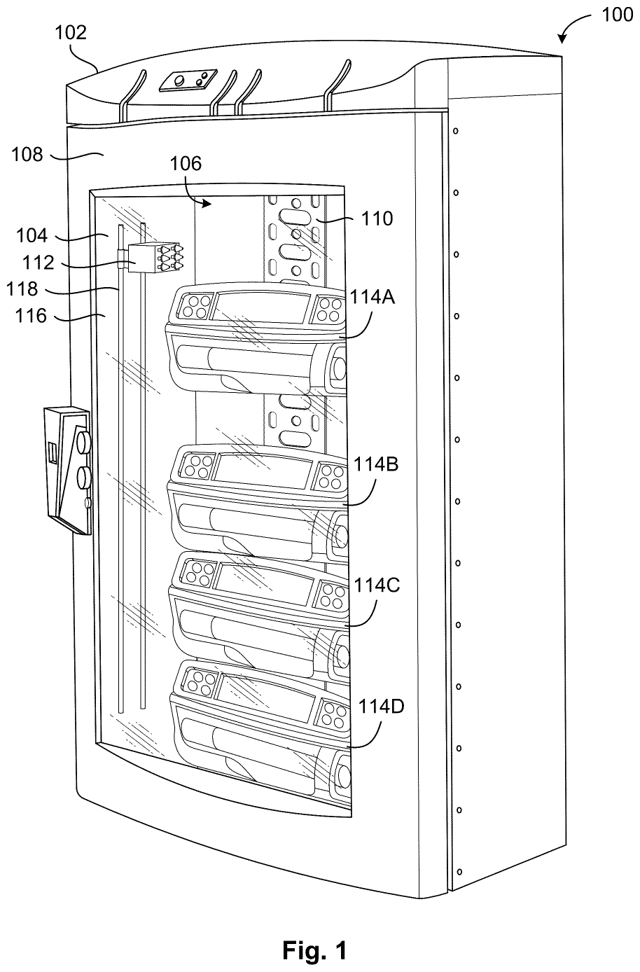

FIG. 1 illustrates an example cabinet 100 for cleaning medical devices 114A-D stored therein, according to various aspects of the subject technology. This cabinet 100 can help streamline medical device cleaning, for instance, by automating cleaning routines, tracking cleaning histories, and reporting cleaning information for better fleet management.

The cabinet 100 includes a housing 102 with an inner surface 104 that defines an enclosure 106 sized to receive one or more medical devices 114A-D. In the illustrated implementation, each of the medical devices 114A-D contained in the cabinet 100 is a syringe pump; however, the cabinet 100 can also receive other types of medical devices, such as patient care units, infusion modules, and the like. For purposes of the present disclosure, “medical device” should be understood as referring to any medical device or medical equipment that requires regular cleaning and can conceivably be received within the cabinet 100.

In addition to the inner surface 104, the housing 102 also includes a door 108 configured to secure the enclosure 106. A technician can open the door 108 to place the medical devices 114A-D into the enclosure 106 or remove the medical devices 114A-D therefrom. According to various implementations, the door 108 forms an airtight seal when it is closed for preventing cleaning agents from unintentionally exiting the cabinet 100. The door 108 can also include a transparent portion for viewing the medical devices 114A-D while the door 108 is closed, as is the case in the illustrated implementation. In some implementations, the door 108 includes a lock that prevents opening of the door 108. The lock may be actuated to further ensure the door 108 is not unintentionally opened so as to cause cleaning agents to exit the cabinet 100.

The cabinet 100 also includes a mechanical retainer 110 configured to retain the medical devices 114A-D while they are received within the enclosure 106. In the illustrated implementation, the mechanical retainer 110 is positioned vertically within the cabinet so that, when the medical devices 114A-C are coupled to the retainer 110, the devices are suspended in a stacked configuration such that one of the devices 114D is suspended over a floor of the enclosure 106 and each of the other medical devices 114A-C is suspended over another medical device.

Moreover, in the illustrated implementation, the retainer 110 is a rail to which respective rear portions of the medical devices 114A-D are fastened. For instance, the medical devices 114A-D can include threaded bolt holes or another interface whereby hardware can be used to affix the medical devices 114A-D to the mechanical retainer 110. The mechanical retainer 110 can also be implemented, for instance, as a latching mechanism, a shelf assembly, or any other mechanical assembly for retaining the medical devices 114A-D while they are within the cabinet 100. Additionally, according to various implementations, the retainer 110 is configured such that the medical devices can be stored vertically or in orientations other than those illustrated in FIG. 1.

Additionally, the cabinet 100 includes a robotic cleaning head 112 configured to apply a cleaning agent (e.g., a light-based agent, heightened temperatures, a chemical agent, a or combination thereof) for cleaning the medical devices 114A-D while they are received within the enclosure 106. In the illustrated implementation, the robotic cleaning head 112 is mounted to a track 118 and configured to traverse the track 118 in order to move within the enclosure 106 and apply the cleaning agent to the medical devices 114A-D. The track 118 is fixedly coupled to a wall 116 of the inner surface 104 and extends along a significant portion (e.g., at least half) of a length of the wall 116. Alternatively, or additionally, in some implementations, the robotic cleaning head 112 can be mounted to a robotic arm (not pictured) or another mechanism configured to move the robotic cleaning head 112 around the enclosure 106.

In some implementations, the robotic cleaning head 112 is affixed to a more sophisticated robotic arm assembly (not pictured). The robotic arm assembly can include a multi-jointed structure with at least two degrees of freedom, allowing the head 112 to achieve precise positioning for better cleaning the medical devices 112A-D. The primary components of the robotic arm assembly can include a base with rotational capability, an articulated arm with an elbow joint, and an end portion affixed to the cleaning head 112. The arm's movement can be actuated by a combination of stepper motors and/or servo motors, which can work in tandem to control both rotational and/or linear motions. Additionally, in some implementations, a feedback system comprising one or more sensors monitors the position of the arm assembly and the state of the head 112, ensuring accurate control and timely error correction. This feedback system can be informed by machine vision or artificial intelligence to ensure that the entirety of the medical devices 114A-D are cleaned.

Additionally, the cabinet 100 can include other robotic cleaning heads in addition to the pictured cleaning head 112, allowing the cabinet 100 to better clean (e.g., more efficiently, more quickly) the medical devices 114A-D therein or allowing the cabinet 100 to clean multiple of the medical devices 114A-D at the same time. Further, the cleaning head 112 can be located at other portions of the cabinet 100 not pictured in FIG. 1 (e.g., affixed to an inner surface of the door 108, affixed to another wall within the enclosure 106). For example, the cleaning head 112 can be connected to a back wall of the enclosure 106 via a robotic arm able to bend at an angle (e.g., 90 degrees) so the cleaning head 112 can reach around the medical devices 114A-D. Moreover, the cleaning head 112 can be capable of rotation and/or both horizontal and vertical movement (e.g., simultaneously).

In some implementations, the cleaning agent is a liquid cleaning agent (e.g., isopropyl alcohol), and the robotic cleaning head 112 includes a sprayer (e.g., one or more spray jets) configured to spray the liquid cleaning agent on the medical devices 114A-D while they are received within the enclosure 106. In such implementations, the cabinet 100 may include a drain for draining the liquid cleaning agent from the housing 102 after the medical devices 114A-D are cleaned. Further, in some implementations, the cleaning agent is a light-based cleaning agent (e.g., blue light, ultraviolet light), and the robotic cleaning head 112 includes a light-based sanitizer configured to shine the light-based cleaning agent on the medical devices 114A-D while they are received within the enclosure 106.

An example workflow involving the cabinet 100 begins with a technician gathering the medical devices 114A-D for cleaning. This may occur in response to a cleaning reminder sent from the cabinet 100 to the technician, or the technician may gather the medical devices 114A-D without a notification. After the technician secures the medical devices 114A-D in the enclosure 106 with the mechanical retainer 110, the technician then closes the door 108 and the cabinet 100 can begin the cleaning process.

At some point before, during, or after cleaning the medical devices 114A-D, the cabinet 100 may receive or retrieve respective identifiers (e.g., serial numbers) for the devices 114A-D. For instance, the technician can scan respective machine-readable codes on the medical devices 114A-D with a scanner (e.g., a barcode scanner, a mobile device) that is communicatively coupled to the cabinet 100. Or the cabinet 100 can identify the medical devices 114A-D via an electrical (e.g., an inter-unit interface (IUI) connector, a universal serial bus (USB) connector) or a wireless connection (e.g., Bluetooth, Near-Field Communication).

The cabinet 100 can use these identifiers to update a cleaning log with cleaning information (e.g., a time of cleaning, a duration of cleaning, a cleaning protocol used) regarding the devices 114A-D. Additionally, or alternatively, the cabinet 100 can use the identifiers to determine one or more parameters for cleaning the medical devices 114A-D, such as (i) one or more cleaning agents (e.g., a light-based agent, heightened temperatures, a chemical agent) to use when cleaning the devices 114A-D, (ii) one or more steps to follow when cleaning the devices 114A-D, (iii) a duration for cleaning the devices 114A-D, or (iv) one or more areas to focus on when cleaning the devices 114A-D. In some implementations, the cabinet 100 can use a medical device identifier to query a cleaning log and determine how long it has been since the medical device corresponding to the identifier was cleaned, what cleaning agent was used when the medical device was cleaned, and other such information. For example, if the cabinet 100 determines that it has been a long time (e.g., more than seven days) since the medical device was last cleaned, the cabinet may spend more time (e.g., an additional five minutes) cleaning the medical device to ensure it is cleaned properly. In some implementations, if a device should only be cleaned using certain agents, the cabinet 100 may receive instructions to clean using prescribed agent(s). As another example, the device identifier may be used to determine usage information for the device within a clinical environment such as where the device was used and how long it was used. Based on usage information, the cabinet 100 may receive cleaning instructions such as how long to clean and which cleaning agent(s) to use.

Additionally, the cabinet 100 may include a camera (not shown). The camera may be used to inspect or document the device cleaning. For example, the device may be imaged prior to cleaning and after cleaning. The before and after images may be compared to determine the effect of the cleaning protocol. In some implementations, the camera may be mounted on or near the door 108 or on or near the cleaning head 112 or similar robotic arm to provide a field of view encompassing the medical devices 114A-D when received in the enclosure 106. In some implementations, the adequacy of the cleaning may be a factor in releasing a lock of the door 108.

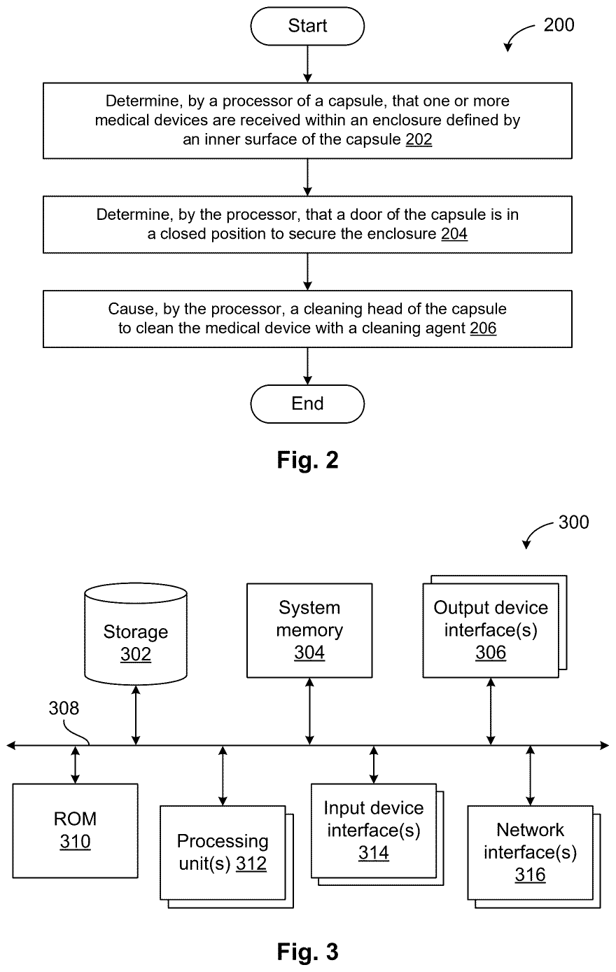

FIG. 2 depicts an example process 200 for cleaning medical devices (e.g., medical devices 114A-D), according to various aspects of the subject technology. For explanatory purposes, the present disclosure describes the blocks of the example process 200 with reference to FIG. 1 and the components described therein. One or more of the blocks of process 200 may be implemented by one or more of computing devices, such as a processor of the cabinet 100 of FIG. 1 or a processor of a server communicatively connected to the cabinet 100.

In some implementations, one or more of the blocks may be implemented based on one or more machine learning algorithms. In some implementations, one or more of the blocks may be implemented apart from other blocks, and by one or more different processors or devices. Further, for explanatory purposes, the blocks of the process 200 are described as occurring in serial, or linearly. However, multiple blocks of the process 200 may occur in parallel. Additionally, the blocks of the process 200 need not be performed in the order shown and one or more of the blocks of the process 200 need not be performed.

In the depicted example, a processor (e.g., a processor of cabinet 100) determines (202) that one or more medical devices (e.g., one or more of medical devices 114A-D) are received within an enclosure (e.g., enclosure 106) sized to receive the one or more medical devices. The enclosure is defined by an inner surface (e.g., inner surface 104) of a housing (e.g., housing 102) of a cabinet (e.g., cabinet 100) for cleaning medical devices (e.g., medical devices 114A-D). In addition to the inner surface, the housing also includes a door (e.g., door 108) configured to secure the enclosure.

Additionally, the cabinet also includes a mechanical retainer (e.g., mechanical retainer 110) positioned on a first surface of the enclosure and configured to immovably couple the one or more medical devices to the first surface of the enclosure while the one or more medical devices are received within the enclosure. Additionally, the cabinet includes a robotic cleaning head (e.g., cleaning head 112) movably coupled to a second surface of the enclosure and configured to move within the enclosure and apply a cleaning agent (e.g., a light-based agent, heightened temperatures, a chemical agent) to the one or more medical devices while they are received within the enclosure.

The processor determining (202) that the medical device is received within the enclosure can be based, for instance, on a user input indicating insertion of the medical device, a reading from a sensor configured to detect displacement of the mechanical retainer, or a communication from the medical device to the processor.

The processor also determines (204) that the door of the capsule is in a closed position to secure the enclosure. Like the first determination (202), this determination (204) can also be based on receipt of user input. However, in some implementations, determining (204) the door is in the closed position is based on a reading from a sensor configured to detect whether the door is in the closed position (or in an open position). In implementations including a lock, the determination may be based on a sensor measurement indicating a state of the lock.

After the processor determines (202) that the medical device is received within the enclosure and also determines (204) that the door is in the closed position, the processor then causes (206) the robotic cleaning head to move within the enclosure and apply the cleaning agent to clean the one or more medical devices. This may involve the robotic cleaning head moving within the enclosure (e.g., by traversing track 118) in order to apply the cleaning agent to the entirety, or at least a substantial portion (e.g., 75%, 85%), of an outer surface of the one or more medical devices. Additionally or alternatively, this may involve activating a sprayer, a light emitter, or another cleaning agent applicator.

In some implementations, the processor updates a digital cleaning log with a time at which the one or more medical devices were cleaned. The cleaning log is a collection of data regarding the cleaning of the medical device and, optionally, other data regarding the cleaning of other medical devices. According to various implementations, updating the cleaning log involves providing to the cleaning log the time at which the one or more medical devices were cleaned, as well as providing to the cleaning log respective identifiers for the one or more medical devices. Additionally, updating the cleaning log may also include providing to the log information regarding the type of cleaning performed (e.g., a standard clean, a deep clean), the cleaning agents used, or other information regarding the manner or degree to which cabinet cleaned the one or more medical devices.

Cleaning log data can be used by the processor to determine various parameters for cleaning the one or more medical devices (e.g., when re-cleaning the medical device at a later time). For instance, before causing the robotic cleaning head to clean the one or more medical devices, the processor can determine a duration for cleaning the one or more medical devices based on an amount of time since the one or more medical devices were last cleaned (e.g., as determined based on cleaning log data). Additionally, or alternatively, the processor can determine the duration based on a type of the one or more medical devices (e.g., or an amount of use of the one or more medical devices since the one or more medical devices were last cleaned (e.g., as determined based on cleaning log data and use history data). In such implementations, causing the robotic cleaning head to clean the one or more medical devices comprises causing the robotic cleaning head to clean the one or more medical devices for the determined duration.

Similarly, in some implementations, the processor is configured to determine one or more focus areas (e.g., a keypad) for cleaning the one or more medical devices before causing the robotic cleaning head to clean the one or more medical devices. Like the duration determination described immediately above, this determination can be based on the amount of time since the one or more medical devices were last cleaned (e.g., as determined based on cleaning log data), a type of the one or more medical devices, or the amount of use of the one or more medical devices since the one or more medical devices were last cleaned (e.g., as determined based on cleaning log data and use history data). In such implementations, causing the robotic cleaning head to clean the one or more medical devices comprises causing the robotic cleaning head to focus on the one or more focus areas while cleaning the one or more medical devices. For example, the robotic cleaning head may spend more time applying the cleaning agent or apply more of the cleaning agent to the one or more focus areas.

In some implementations, the cabinet further includes one or more electrical couplers (e.g., IUI connectors, USB connectors) configured to provide electrical power to the one or more medical devices while the one or more medical devices are received within the enclosure. The electrical coupler can be further configured to permit the processor to communicate with the one or more medical devices while the one or more medical devices are received within the enclosure. According to various implementations, this allows the processor to perform a firmware update for the one or more medical devices while they are being cleaned. For example, the processor can retrieve one or more firmware versions of firmware running on the one or more medical device and then determine based on the firmware version(s) whether the firmware running on the one or more medical devices is out of date. If it is, the processor can provide the one or more medical devices with a firmware update accordingly.

Relatedly, according to various implementations, the cabinet can be configured to perform maintenance or calibration functions for the one or more medical devices received therein. For example, after the one or more medical devices are received into the cabinet, the cabinet can connect to the one or more medical devices (e.g., via electrical couplers) and perform software updates for the one or more medical devices and/or recalibrate the one or more medical devices. Likewise, reporting for the one or more medical devices can be performed by the medical cabinet. For example, if the cabinet determines one of the medical devices requires technician attention (e.g., due to software update issues, calibration issues), the cabinet can alert a technician regarding identified issues.

The determination may be based on usage information received based, at least in part, on the device identifier. For example, the device may be associated with a manufacturing lot or date that requires calibration. As another example, it may be known that exceeding a certain number of infusions or certain number of hours of infusing may require a device to be inspected. The cabinet 100 may transmit and measure signals to the device and, based on those measurements, determine the device needs additional adjustment or maintenance. For example, if the signal strength falls below a threshold, the device may need servicing.

An example of a calibration may include color calibration of a display of the device. Over time, displays may experience shift in how a color is presented. This can cause a display of a device to render colors that are not aligned with the specification or directions for use. In instances of the described technology, once cleaned, color information for the display may be obtained (e.g., via a camera). The cabinet 100 may then review the color information and transmit adjustments to the device to ensure the colors are displayed according to a predetermined scheme (e.g., manufacturer specification).

As noted above, in some implementations, the cleaning agent is a liquid cleaning agent and the robotic cleaning head comprises a sprayer configured to spray the liquid cleaning agent on the medical device while the one or more medical devices are received within the enclosure. In such implementations, the processor can be configured to, before causing the robotic cleaning head to clean the one or more medical devices, determine an amount of the liquid cleaning agent to use when cleaning the one or more medical devices. This determination can be based on an amount of time since the one or more medical devices were last cleaned (e.g., as determined based on cleaning log data), a type of the one or more medical devices, or an amount of use of the one or more medical devices since the one or more medical devices were last cleaned (e.g., as determined based on cleaning log data and use history data). In such implementations, causing the robotic cleaning head to clean the one or more medical devices comprises causing the robotic cleaning head to spray the determined amount of the liquid cleaning agent on the one or more medical devices.

The cabinet can also be configured to clean multiple medical devices at the same time. In some implementations, the enclosure is sized to receive a first medical device (e.g., medical device 114D) and a second medical device (e.g., medical device 114C). Accordingly, the processor can be further configured to determine that the first and second medical devices are received within the enclosure and cause the robotic cleaning head to clean the first and second medical devices (e.g., at the same time) with the cleaning agent after determining that the first and second medical devices are received within the enclosure. The cabinet can also include multiple robotic cleaning heads to allow it to apply the cleaning agent simultaneously to multiple different medical devices or to allow it to apply different cleaning agents simultaneously to one or more of the medical devices.

FIG. 3 illustrates an example electronic system 300 for cleaning medical devices, according to various aspects of the subject technology. Electronic system 300 may be implemented by a computing device for execution of software associated with portions or steps of process 200 of FIG. 2, or components provided by FIG. 1. In this regard, the electronic system 300 may include the cabinet 100 of FIG. 1 or a server connected thereto.

The electronic system 300 may also include a specifically-configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Additionally, the electronic system 300 may include various types of computer-readable media and interfaces for various other types of computer-readable media. In the depicted example, electronic system 300 includes a bus 308, a processing unit(s) 312, a system memory 304, a read-only memory (ROM) 310, a permanent storage device 302, an input device interface(s) 314, an output device interface(s) 306, and a network interface(s) 316. In some implementations, electronic system 300 may include or be integrated with other computing devices or circuitry for operation of the various components and methods previously described.

Bus 308 collectively represents system, peripheral, and chipset buses that communicatively connect the numerous internal devices of electronic system 300. For instance, bus 308 communicatively connects processing unit(s) 312 with ROM 310, the system memory 304, and permanent storage device 302. From these various memory units, processing unit(s) 312 retrieves instructions to execute and data to process in order to execute the processes of the subject disclosure. Processing unit(s) 312 can be a single processor or a multi-core processor in different implementations.

ROM 310 stores static data and instructions that are needed by processing unit(s) 312 and other modules of the electronic system. Permanent storage device 302, on the other hand, is a read-and-write memory device. This device is a non-volatile memory unit that stores instructions and data even when electronic system 300 is powered off. Some implementations of the subject disclosure use a mass-storage device (such as a magnetic or optical disk and its corresponding disk drive) as permanent storage device 302. Other implementations use a removable storage device (such as a floppy disk, flash drive, and its corresponding disk drive) as permanent storage device 302.

Like permanent storage device 302, system memory 304 is a read-and-write memory device. However, unlike storage device 302, system memory 304 is a volatile read-and-write memory, such as random-access memory (RAM). System memory 304 stores some of the instructions and data that the processor needs at runtime. In some implementations, the processes of the subject disclosure are stored in system memory 304, permanent storage device 302, and/or ROM 310. From these various memory units, processing unit(s) 312 retrieves instructions to execute and data to process, in order to execute the processes of some implementations.

Bus 308 also connects to input device interface(s) 314 and output device interface(s) 306. Input device interface(s) 314 enables the user to communicate information and select commands to the electronic system. Input devices used with input device interface(s) 314 include, for example, alphanumeric keyboards and pointing devices (also called “cursor control devices”). Output device interface(s) 306 enables, for example, the display of images generated by electronic system 300. Output devices used with output device interface(s) 306 include, for example, printers and display devices, such as cathode ray tubes (CRT) or liquid crystal displays (LCD). Some implementations include devices (e.g., touchscreens) that function as both input and output devices.

Furthermore, bus 308 also couples electronic system 300 to a network (not shown) through network interface(s) 316. Network interface(s) 316 may include, for example, a wireless access point (e.g., Bluetooth or Wi-Fi) or radio circuitry for connecting to a wireless access point. Network interface(s) 316 may also include hardware (e.g., ethernet hardware) for connecting the computer to a part of a network of computers such as a local area network (LAN), a wide area network (WAN), wireless LAN, an intranet, or a network of networks, such as the Internet. Components of electronic system 300 can be used in conjunction with the subject disclosure when specifically configured with one of more of the features described.

The functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer-readable medium” and “computer-readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a LAN and a WAN, an inter-network (e.g., Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Illustrative Clauses. For further reference, example aspects of the present disclosure are included below as numbered clauses. These clauses are provided for illustrative purposes and are not intended to limit the subject technology.

Clause 1. A cabinet for cleaning medical devices, the cabinet comprising: a housing comprising (i) an enclosure sized to receive one or more medical devices and (ii) a door configured to secure the enclosure; a mechanical retainer positioned on a first surface of the enclosure and configured to immovably couple the one or more medical devices to the surface of the enclosure while the one or more medical devices are received within the enclosure; a robotic cleaning head movably coupled to a second surface of the enclosure and configured to move within the enclosure and apply a cleaning agent to the one or more medical devices while the one or more medical devices are received within the enclosure; and a processor configured to (i) determine that the one or more medical devices are received within the enclosure, (ii) determine that the door is in a closed position to secure the enclosure, and (iii) cause the robotic cleaning head to move within the enclosure and apply the cleaning agent to clean the one or more medical devices after determining that the one or more medical devices are received within the enclosure and the door is in the closed position.

Clause 2. The cabinet of clause 1, wherein the robotic cleaning head is configured to traverse a track that is vertically positioned within the enclosure.

Clause 3. The cabinet of clause 2, wherein the track is fixedly coupled to another surface of the enclosure with a length of the track traversing at least half of a length of the enclosure.

Clause 4. The cabinet of any one of clauses 1 through 3, wherein: the processor is further configured to, after causing the robotic cleaning head to clean the one or more medical devices, update a digital cleaning log with a time at which the one or more medical devices were cleaned.

Clause 5. The cabinet of any one of clauses 1 through 4, wherein: the processor is further configured to, before causing the robotic cleaning head to clean the one or more medical devices, determine a duration for cleaning the one or more medical devices based on (i) an amount of time since the one or more medical devices were last cleaned or (ii) an amount of use of the one or more medical devices since the one or more medical devices were last cleaned; and causing the robotic cleaning head to clean the one or more medical devices comprises causing the robotic cleaning head to clean the one or more medical devices for the determined duration.

Clause 6. The cabinet of any one of clauses 1 through 5, wherein: the processor is further configured to, before causing the robotic cleaning head to clean the one or more medical devices, determine one or more focus areas for cleaning the one or more medical devices based on (i) a type of the one or more medical devices or (ii) a use history of the one or more medical device; and causing the robotic cleaning head to clean the one or more medical devices comprises causing the robotic cleaning head to focus on the one or more focus areas while cleaning the one or more medical devices.

Clause 7. The cabinet of any one of clauses 1 through 6, further comprising: one or more electrical couplers configured to provide electrical power from the cabinet to the one or more medical devices while the one or more medical devices are received within the enclosure.

Clause 8. The cabinet of clause 7, wherein: the one or more electrical couplers are further configured to permit the processor to communicate with the one or more medical devices while the one or more medical devices are received within the enclosure; and the processor is further configured to: retrieve a firmware version of firmware running on one of the one or more medical devices; determine based on the firmware version that the firmware running on the one or more medical devices is out of date; and provide the one or more medical devices with a firmware update responsive to determining that the firmware is out of date.

Clause 9. The cabinet of any one of clauses 1 through 8, wherein: the cleaning agent is a liquid cleaning agent; the robotic cleaning head comprises a sprayer configured to spray the liquid cleaning agent on the one or more medical devices while the one or more medical devices are received within the enclosure; and the housing further comprises a drain configured to allow the liquid cleaning agent to drain from the housing after the sprayer sprays the liquid cleaning agent on the one or more medical devices.

Clause 10. The cabinet of clause 9, wherein: the processor is further configured to, before causing the robotic cleaning head to clean the one or more medical devices, determine an amount of the liquid cleaning agent to use when cleaning the one or more medical devices based on (i) an amount of time since the one or more medical devices were last cleaned or (ii) an amount of use of the one or more medical devices since the one or more medical devices were last cleaned; and causing the robotic cleaning head to move within the enclosure and apply the cleaning agent comprises causing the robotic cleaning head to spray the determined amount of the liquid cleaning agent on the one or more medical devices.

Clause 11. The cabinet of any one of clauses 1 through 10, wherein: the cleaning agent comprises ultraviolet light; and the robotic cleaning head comprises an ultraviolet light sanitizer configured to shine ultraviolet light on the one or more medical devices while the one or more medical devices are received within the enclosure.

Clause 12. The cabinet of any one of clauses 1 through 11, wherein: the one or more medical devices comprises a first medical device and a second medical device; and determining that the one or more medical devices are received within the enclosure comprises determining that the first and second medical devices are received within the enclosure.

Clause 13. The cabinet of clause 12, wherein: the enclosure is further sized to receive additional medical devices; determining that the one or more medical devices are received within the enclosure further comprises determining that the additional medical devices are received within the enclosure; and the mechanical retainer is positioned vertically within the cabinet so that, when the one or more medical devices are coupled to the mechanical retainer, the medical devices are suspended in a stacked configuration such that the first medical device is suspended over a floor of the enclosure and each of the second and additional medical devices are suspended over another medical device.

Clause 14. A computer-implemented method for cleaning medical devices, the method comprising: determining, by a processor of a cabinet, that one or more medical devices are received within an enclosure sized to receive the one or more medical devices, wherein the cabinet further comprises: a housing comprising (i) the enclosure and (ii) a door configured to secure the enclosure; a mechanical retainer positioned on a first surface of the enclosure and configured to immovably couple the one or more medical devices to the first surface of the enclosure while the one or more medical devices are received within the enclosure; and a robotic cleaning head movably coupled to a second surface of the enclosure and configured to move within the enclosure and apply a cleaning agent to the one or more medical devices while the one or more medical devices are received within the enclosure; determining, by the processor, that the door is in a closed position to secure the enclosure; and causing, by the processor, the robotic cleaning head to move within the enclosure and apply the cleaning agent to clean the one or more medical devices after determining that the one or more medical devices are received within the enclosure and the door is in the closed position.

Clause 15. The computer-implemented method of clause 14, further comprising: after causing the robotic cleaning head to clean the one or more medical devices, updating, by the processor, a digital cleaning log with a time at which the one or more medical devices were cleaned.

Clause 16. The computer-implemented method of any one of clauses 14 through 15, further comprising: before causing the robotic cleaning head to clean the one or more medical devices, determining, by the processor, a duration for cleaning the one or more medical devices based on (i) an amount of time since the one or more medical devices were last cleaned or (ii) an amount of use of the one or more medical devices since the one or more medical devices were last cleaned, wherein causing the robotic cleaning head to clean the one or more medical devices comprises causing the robotic cleaning head to clean the one or more medical devices for the determined duration; or before causing the robotic cleaning head to clean the one or more medical devices, determining, by the processor, one or more focus areas for cleaning the one or more medical devices based on (i) a type of the one or more medical devices or (ii) a use history of the one or more medical devices, wherein causing the robotic cleaning head to clean the one or more medical devices comprises causing the robotic cleaning head to focus on the one or more focus areas while cleaning the one or more medical devices.

Clause 17. The computer-implemented method of any one of clauses 14 through 16, further comprising: retrieving, by the processor, a firmware version of firmware running on the one or more medical devices via one or more electrical couplers of the cabinet, wherein the one or more electrical couplers are configured to (i) provide electrical power from the cabinet to the one or more medical devices while the one or more medical devices are received within the enclosure and (ii) permit the processor to communicate with the one or more medical devices while the one or more medical devices are received within the enclosure; determining, by the processor and based on the firmware version, that the firmware running on the one or more medical devices is out of date; and providing, by the processor, the one or more medical devices with a firmware update responsive to determining that the firmware is out of date.

Clause 18. The computer-implemented method of any one of clauses 14 through 17, further comprising: before causing the robotic cleaning head to clean the one or more medical devices, determining, by the processor, an amount of a liquid cleaning agent to use when cleaning the one or more medical devices based on (i) an amount of time since the one or more medical devices were last cleaned or (ii) an amount of use of the one or more medical devices since the one or more medical devices were last cleaned; wherein the cleaning agent is a liquid cleaning agent; wherein the robotic cleaning head comprises a sprayer configured to spray the liquid cleaning agent on the one or more medical devices while the one or more medical devices are received within the enclosure; wherein causing the robotic cleaning head to move within the enclosure and apply the cleaning agent comprises causing the robotic cleaning head to spray the determined amount of the liquid cleaning agent on the medical device; and wherein the housing further comprises a drain configured to allow the liquid cleaning agent to drain from the housing after the sprayer sprays the liquid cleaning agent on the one or more medical devices.

Clause 19. The computer-implemented method of any one of clauses 14 through 18, further comprising: determining that one or more medical devices are received within the enclosure comprises determining that a first medical device, a second medical device, and additional medical devices are received within the enclosure; wherein the one or more medical devices comprises the first medical device, the second medical device, and the additional medical devices; and wherein the mechanical retainer is positioned vertically within the cabinet so that, when the one or more medical devices are coupled to the mechanical retainer, the medical devices are suspended in a stacked configuration such that the first medical device is suspended over a floor of the enclosure and each of the second and additional medical devices are suspended over another medical device.

Clause 20. A non-transitory, computer-readable storage medium comprising instructions that, when executed by a processor of a cabinet for cleaning medical devices, cause the cabinet to: determine that one or more medical devices are received within an enclosure sized to receive the one or more medical devices, wherein the cabinet further comprises: a housing comprising (i) the enclosure and (ii) a door configured to secure the enclosure; a mechanical retainer positioned on a first surface of the enclosure and configured to immovably couple the one or more medical devices to the first surface of the enclosure while the one or more medical devices are received within the enclosure; and a robotic cleaning head movably coupled to a second surface of the enclosure and configured to move within the enclosure and apply a cleaning agent to the one or more medical devices while the one or more medical devices are received within the enclosure; determine that the door is in a closed position to secure the enclosure; and cause the robotic cleaning head to move within the enclosure and apply the cleaning agent to clean the one or more medical devices after determining that the one or more medical devices are received within the enclosure and the door is in the closed position.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be appreciated that a message may be composed, transmitted, stored, received, and so on in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine-learning assessment model, or combinations thereof.

In any implementation, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A cabinet for cleaning medical devices, the cabinet comprising:
a housing comprising (i) an enclosure sized to receive one or more medical devices and (ii) a door configured to secure the enclosure;
a mechanical retainer positioned on a first surface of the enclosure and configured to immovably couple the one or more medical devices to the surface of the enclosure while the one or more medical devices are received within the enclosure;
a robotic cleaning head movably coupled to a second surface of the enclosure and configured to move within the enclosure and apply a cleaning agent to the one or more medical devices while the one or more medical devices are received within the enclosure; and
a processor configured to (i) determine that the one or more medical devices are received within the enclosure, (ii) determine that the door is in a closed position to secure the enclosure, and (iii) cause the robotic cleaning head to move within the enclosure and apply the cleaning agent to clean the one or more medical devices after determining that the one or more medical devices are received within the enclosure and the door is in the closed position.

2. The cabinet of claim 1, wherein the robotic cleaning head is configured to traverse a track that is vertically positioned within the enclosure.

3. The cabinet of claim 2, wherein the track is fixedly coupled to another surface of the enclosure with a length of the track traversing at least half of a length of the enclosure.

4. The cabinet of claim 1, wherein:
the processor is further configured to, after causing the robotic cleaning head to clean the one or more medical devices, update a digital cleaning log with a time at which the one or more medical devices were cleaned.

5. The cabinet of claim 1, wherein:
the processor is further configured to, before causing the robotic cleaning head to clean the one or more medical devices, determine a duration for cleaning the one or more medical devices based on (i) an amount of time since the one or more medical devices were last cleaned or (ii) an amount of use of the one or more medical devices since the one or more medical devices were last cleaned; and
causing the robotic cleaning head to clean the one or more medical devices comprises causing the robotic cleaning head to clean the one or more medical devices for the determined duration.

6. The cabinet of claim 1, wherein:
the processor is further configured to, before causing the robotic cleaning head to clean the one or more medical devices, determine one or more focus areas for cleaning the one or more medical devices based on (i) a type of the one or more medical devices or (ii) a use history of the one or more medical device; and
causing the robotic cleaning head to clean the one or more medical devices comprises causing the robotic cleaning head to focus on the one or more focus areas while cleaning the one or more medical devices.

7. The cabinet of claim 1, further comprising:
one or more electrical couplers configured to provide electrical power from the cabinet to the one or more medical devices while the one or more medical devices are received within the enclosure.

8. The cabinet of claim 7, wherein:
the one or more electrical couplers are further configured to permit the processor to communicate with the one or more medical devices while the one or more medical devices are received within the enclosure; and
the processor is further configured to:
retrieve a firmware version of firmware running on one of the one or more medical devices;
determine based on the firmware version that the firmware running on the one or more medical devices is out of date; and
provide the one or more medical devices with a firmware update responsive to determining that the firmware is out of date.

9. The cabinet of claim 1, wherein:
the cleaning agent is a liquid cleaning agent;
the robotic cleaning head comprises a sprayer configured to spray the liquid cleaning agent on the one or more medical devices while the one or more medical devices are received within the enclosure; and
the housing further comprises a drain configured to allow the liquid cleaning agent to drain from the housing after the sprayer sprays the liquid cleaning agent on the one or more medical devices.

10. The cabinet of claim 9, wherein:
the processor is further configured to, before causing the robotic cleaning head to clean the one or more medical devices, determine an amount of the liquid cleaning agent to use when cleaning the one or more medical devices based on (i) an amount of time since the one or more medical devices were last cleaned or (ii) an amount of use of the one or more medical devices since the one or more medical devices were last cleaned; and
causing the robotic cleaning head to move within the enclosure and apply the cleaning agent comprises causing the robotic cleaning head to spray the determined amount of the liquid cleaning agent on the one or more medical devices.

11. The cabinet of claim 1, wherein:
the cleaning agent comprises ultraviolet light; and
the robotic cleaning head comprises an ultraviolet light sanitizer configured to shine ultraviolet light on the one or more medical devices while the one or more medical devices are received within the enclosure.

12. The cabinet of claim 1, wherein:
the one or more medical devices comprises a first medical device and a second medical device; and
determining that the one or more medical devices are received within the enclosure comprises determining that the first and second medical devices are received within the enclosure.

13. The cabinet of claim 12, wherein:
the enclosure is further sized to receive additional medical devices;
determining that the one or more medical devices are received within the enclosure further comprises determining that the additional medical devices are received within the enclosure; and
the mechanical retainer is positioned vertically within the cabinet so that, when the one or more medical devices are coupled to the mechanical retainer, the medical devices are suspended in a stacked configuration such that the first medical device is suspended over a floor of the enclosure and each of the second and additional medical devices are suspended over another medical device.

14. A computer-implemented method for cleaning medical devices, the method comprising:
determining, by a processor of a cabinet, that one or more medical devices are received within an enclosure sized to receive the one or more medical devices, wherein the cabinet further comprises:
a housing comprising (i) the enclosure and (ii) a door configured to secure the enclosure;
a mechanical retainer positioned on a first surface of the enclosure and configured to immovably couple the one or more medical devices to the first surface of the enclosure while the one or more medical devices are received within the enclosure; and
a robotic cleaning head movably coupled to a second surface of the enclosure and configured to move within the enclosure and apply a cleaning agent to the one or more medical devices while the one or more medical devices are received within the enclosure;
determining, by the processor, that the door is in a closed position to secure the enclosure; and
causing, by the processor, the robotic cleaning head to move within the enclosure and apply the cleaning agent to clean the one or more medical devices after determining that the one or more medical devices are received within the enclosure and the door is in the closed position.

15. The computer-implemented method of claim 14, further comprising:
after causing the robotic cleaning head to clean the one or more medical devices, updating, by the processor, a digital cleaning log with a time at which the one or more medical devices were cleaned.

16. The computer-implemented method of claim 14, further comprising:
before causing the robotic cleaning head to clean the one or more medical devices, determining, by the processor, a duration for cleaning the one or more medical devices based on (i) an amount of time since the one or more medical devices were last cleaned or (ii) an amount of use of the one or more medical devices since the one or more medical devices were last cleaned, wherein causing the robotic cleaning head to clean the one or more medical devices comprises causing the robotic cleaning head to clean the one or more medical devices for the determined duration; or
before causing the robotic cleaning head to clean the one or more medical devices, determining, by the processor, one or more focus areas for cleaning the one or more medical devices based on (i) a type of the one or more medical devices or (ii) a use history of the one or more medical devices, wherein causing the robotic cleaning head to clean the one or more medical devices comprises causing the robotic cleaning head to focus on the one or more focus areas while cleaning the one or more medical devices.

17. The computer-implemented method of claim 14, further comprising:
retrieving, by the processor, a firmware version of firmware running on the one or more medical devices via one or more electrical couplers of the cabinet, wherein the one or more electrical couplers are configured to (i) provide electrical power from the cabinet to the one or more medical devices while the one or more medical devices are received within the enclosure and (ii) permit the processor to communicate with the one or more medical devices while the one or more medical devices are received within the enclosure;
determining, by the processor and based on the firmware version, that the firmware running on the one or more medical devices is out of date; and
providing, by the processor, the one or more medical devices with a firmware update responsive to determining that the firmware is out of date.

18. The computer-implemented method of claim 14, further comprising:
before causing the robotic cleaning head to clean the one or more medical devices, determining, by the processor, an amount of a liquid cleaning agent to use when cleaning the one or more medical devices based on (i) an amount of time since the one or more medical devices were last cleaned or (ii) an amount of use of the one or more medical devices since the one or more medical devices were last cleaned;
wherein the cleaning agent is a liquid cleaning agent;
wherein the robotic cleaning head comprises a sprayer configured to spray the liquid cleaning agent on the one or more medical devices while the one or more medical devices are received within the enclosure;
wherein causing the robotic cleaning head to move within the enclosure and apply the cleaning agent comprises causing the robotic cleaning head to spray the determined amount of the liquid cleaning agent on the medical device; and
wherein the housing further comprises a drain configured to allow the liquid cleaning agent to drain from the housing after the sprayer sprays the liquid cleaning agent on the one or more medical devices.

19. The computer-implemented method of claim 14, further comprising:
determining that one or more medical devices are received within the enclosure comprises determining that a first medical device, a second medical device, and additional medical devices are received within the enclosure;
wherein the one or more medical devices comprises the first medical device, the second medical device, and the additional medical devices; and
wherein the mechanical retainer is positioned vertically within the cabinet so that, when the one or more medical devices are coupled to the mechanical retainer, the medical devices are suspended in a stacked configuration such that the first medical device is suspended over a floor of the enclosure and each of the second and additional medical devices are suspended over another medical device.

20. A non-transitory, computer-readable storage medium comprising instructions that, when executed by a processor of a cabinet for cleaning medical devices, cause the cabinet to:
determine that one or more medical devices are received within an enclosure sized to receive the one or more medical devices, wherein the cabinet further comprises:
a housing comprising (i) the enclosure and (ii) a door configured to secure the enclosure;
a mechanical retainer positioned on a first surface of the enclosure and configured to immovably couple the one or more medical devices to the first surface of the enclosure while the one or more medical devices are received within the enclosure; and
a robotic cleaning head movably coupled to a second surface of the enclosure and configured to move within the enclosure and apply a cleaning agent to the one or more medical devices while the one or more medical devices are received within the enclosure;
determine that the door is in a closed position to secure the enclosure; and
cause the robotic cleaning head to move within the enclosure and apply the cleaning agent to clean the one or more medical devices after determining that the one or more medical devices are received within the enclosure and the door is in the closed position.
